(19) United States
(12) Patent Application Publication
(10) Pub. No.: US 2019/0029495 A1
(43) Pub. Date: Jan. 31, 2019

(54) MEDICAL DEVICE AND THERMOSETTING ADHESIVE FOR MEDICAL DEVICE

(71) Applicant: OLYMPUS CORPORATION, Tokyo (JP)

(72) Inventor: Hiroki YOKOYAMA, Aizu-Wakamatsu-shi (JP)

(73) Assignee: OLYMPUS CORPORATION, Tokyo (JP)

(21) Appl. No.: 16/150,792

(22) Filed: Oct. 3, 2018

Related U.S. Application Data
(63) Continuation of application No. PCT/JP2017/011746, filed on Mar. 23, 2017.

(30) Foreign Application Priority Data
Apr. 7, 2016 (JP) 2016-077397

(51) Int. Cl. A61B 1/00; C08G 59/56; C08L 63/00

(52) U.S. Cl. CPC A61B 1/0011; C08G 59/56; C09J 133/00; A61B 1/0014; A61B 1/00121; C08L 63/00

(57) ABSTRACT

This medical device includes a bonded member, and a cured adhesive body in close contact with the bonded member, wherein the cured adhesive body is formed by curing a thermosetting adhesive for a medical device, and wherein the thermosetting adhesive for a medical device is formed by diffusing an infrared absorbent into a thermosetting resin.

This application is a continuation application based on a PCT International Application No. PCT/JP2017/011746, filed on Mar. 23, 2017, whose priority is claimed on a Japanese Patent Application No. 2016-077397, filed on Apr. 7, 2016. The contents of both the PCT International Application and the Japanese Patent Application are incorporated herein by reference.

### BACKGROUND OF THE INVENTION

### Field of the Invention

The present invention relates to a medical device and a thermosetting adhesive for a medical device.

### Description of Related Art

Since a medical device is used inside the body of a patient and comes in contact with living tissues, an outer surface of the medical device is often sealed to be liquid-tight. Furthermore, as the medical device is immersed into a liquid chemical and exposed in a high-temperature environment for sterilization, the liquid-tight sealed part is often sealed by a thermosetting resin material having a chemical resistance.

An endoscope apparatus is configured by winding tight binding threads on a flexible coating tube formed from an elastomer at a connection portion connecting the coating tube and a rigid member such as an adaptor and the like. The endoscope apparatus is configured to have a liquid-tight fixed portion by binding the coating tube to the rigid member. Furthermore, on the fixed portion, a cured adhesive body is formed by curing a thermosetting adhesive that is coated to cover the coating tube and the tight binding threads. The cured adhesive body comes in close contact with the coating tube and the tight binding threads such that it is possible to achieve a stable fixation and liquid-tight characteristic by the tight binding threads and a smooth insertion of the endoscope apparatus into the body of the patient.

In Japanese Unexamined Patent Application, First Publication No. 2006-218102, an endoscope apparatus configured to have members joined by an epoxy resin adhesive is disclosed.

### SUMMARY OF THE INVENTION

According to a first aspect of the present invention, a medical device includes a bonded member; and a cured adhesive body in close contact with the bonded member, wherein the cured adhesive body is formed by curing a thermosetting adhesive for a medical device, and the thermosetting adhesive for a medical device is formed by diffusing an infrared absorbent into a thermosetting resin.

According to a second aspect of the present invention, in the medical device according to the first aspect, the bonded member may include a flexible coating member configured to coat a base member for fixing; and tight binding threads wound on an outer surface of the coating member. The cured adhesive body may be configured to cover and come in close contact with the tight binding threads and the outer surface of the coating member.

According to a third aspect of the present invention, in the medical device according to the first or the second aspect, the infrared absorbent may include at least one of a group consisting of a cyanine compound, a phthalocyanine compound, a dithiol metal complex, a naphthoquinone compound, a diimonium compound, and an azo compound.

According to a fourth aspect of the present invention, a thermosetting adhesive for a medical device being cured by an irradiation of infrared light includes a thermosetting resin and an infrared absorbent diffused into the thermosetting resin.

According to a fifth aspect of the present invention, in the thermosetting adhesive for a medical device according to the fourth aspect, the infrared absorbent may include at least one of a group consisting of a cyanine compound, a phthalocyanine compound, a dithiol metal complex, a naphthoquinone compound, a diimonium compound, and an azo compound.

### BRIEF DESCRIPTION OF DRAWINGS

FIG. 1 is a schematic partial cross-sectional view showing configurations of main parts of a medical device according to an embodiment of the present invention.

FIG. 2 is a schematic partial cross-sectional view showing a coating procedure of a thermosetting adhesive for a medical device during manufacture of the medical device according to the embodiment of the present invention.

FIG. 3 is a schematic partial cross-sectional view showing a curing procedure of the thermosetting adhesive for a medical device during manufacture of the medical device according to the embodiment of the present invention.

### DETAILED DESCRIPTION OF THE INVENTION

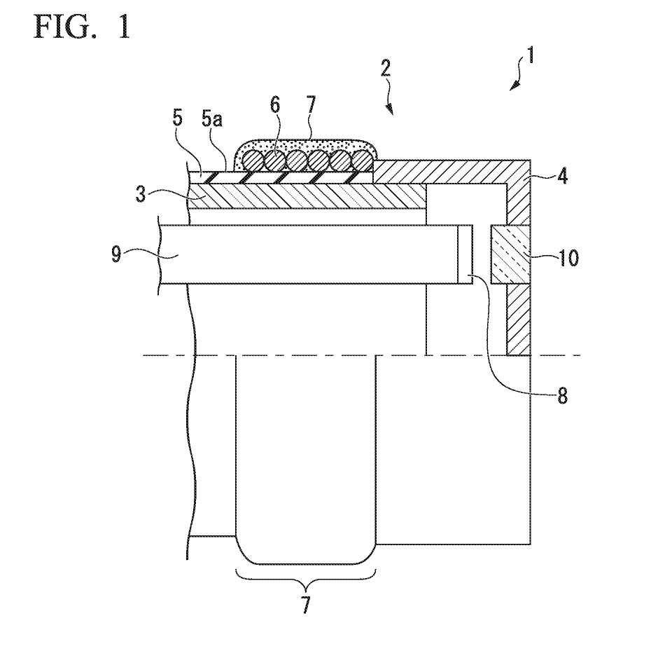

An embodiment of the present invention will be described in accordance with figures. A configuration of a medical device according to the embodiment of the present invention will be described. FIG. 1 is a schematic partial cross-sectional view showing configurations of main parts of the medical device according to the embodiment of the present invention.

As the main parts shown in FIG. 1, an endoscope apparatus (medical device) 1 according to the present embodiment has an insertion portion 2 inserted into the body of a patient and an operation portion provided at a proximal end part of the insertion portion 2 (not shown).

A distal end part of the insertion portion 2 has an adaptor (base member for fixing) 3, a distal-end cover 4, a coating tube (bonded member, coating member) 5, tight binding threads (bonded member) 6, and a cured adhesive body 7.

The adaptor 3 is a cylindrical member disposed at the distal end part of the insertion portion 2 (right side shown in FIG. 1). At a distal end of the adaptor 3, a distal-end cover 4 which will be described later is externally fitted and fixed to the adaptor 3.

At a proximal end (not shown) of the adaptor 3, for example, bending joints and coating tubes configured to cover the bending joints which form a bending portion of the endoscope apparatus 1 are connected to the adaptor 3.

In a center part of the adaptor 3, an opening is formed to pass through the adaptor 3 in a longitudinal direction such that various members inserted into the insertion portion 2 in the longitudinal direction are insertable into the opening.

The distal-end cover 4 is a cylindrical member with a bottom. The distal-end cover 4 is externally fitted with the distal end part of the adaptor 3. An outer surface of the bottom of the distal-end cover 4 is configured to form a distal end surface of the insertion portion 2.

The bottom and a side surface of the distal-end cover 4 may be formed with suitable openings as necessary. For example, when the endoscope apparatus 1 has a treatment tool channel, an opening connecting the distal end opening of the treatment tool channel is formed at the bottom which forms the distal end surface of the distal-end cover 4.

The distal-end cover 4 is configured to liquid-tightly seal the distal end part of the insertion portion 2 except for the opening formed as necessary.

A window portion having optical transparency is formed from a transparent cover at the distal end surface of the distal-end cover 4. At an inward side of at least one window portion, an imaging optical system 10 for guiding light from the outside into the insertion portion 2 is disposed. Since FIG. 1 is a schematic view, an example in which the imaging optical system 10 is a member also serves as the transparent cover is shown. However, the transparent cover and the imaging optical system 10 may be different members, and the imaging optical system 10 may be configured by a combination of a plurality of lens.

At the inward side of the distal-end cover 4, an image sensor 8 is disposed at a position facing the imaging optical system 10, and the image sensor 8 is configured to receive the light passing through the imaging optical system 10 for imaging.

For example, a CCD sensor or a CMOS sensor may be used as the image sensor 8.

The image sensor 8 is electrically connected to a wiring portion 9 via a driving circuit (not shown). The driving circuit (not shown) may be disposed in the distal end part of the insertion portion 2, and the driving circuit may be disposed in the operation portion at the proximal end side thereof.

The wiring portion 9 is configured to have a plurality of wirings which are coated by an insulated coating tube, wherein the plurality of wirings include signal transmission lines for transmitting at least the image signal of the image sensor 8. The wiring portion 9 is inserted into the insertion portion 2 and is extended to the proximal end side of the insertion portion 2.

Although not shown in figures, an illumination light source or a light guide configured to emit illumination light from the illumination window portion provided at the distal end surface of the distal-end cover 4 may be disposed inside the adaptor 3 and the distal-end cover 4. For example, when the illumination light source such as an LED light source is disposed, an LED driving circuit may be disposed at the inward side of the adaptor 3 and the distal-end cover 4.

The coating tube 5 is a tubular member configured to cover an outer circumference of the insertion portion 2 except for the distal end part of the insertion portion 2.

The coating tube 5 is formed from a material having flexibility such that the insertion portion 2 can be bent. For example, the coating tube 5 may be formed from an elastomer having flexibility.

For example, silicone rubber, fluororubber, chloroprene rubber, and a rubber composite including at least one of the materials described above are considered to be suitable materials for forming the coating tube 5. Furthermore, polyether ether ketone resin (PEEK), polyacetal resin (POM), sulfone resin such as the RADEL (registered trade-mark) polyphenyl sulfone and the like, polysulfone resin, and vinyl resin are considered to be suitable materials for forming the coating tube 5.

The tight binding threads 6 are fixing members configured to fix the adaptor 3 by binding the coating tube 5 that is externally fitted with the adaptor 3.

The tight binding threads 6 are wound on an outer circumferential surface (outer surface) 5a of the coating tube 5. An end of the tight binding threads 6 is fixed by a method of inserting the end of the tight binding threads 6 into the space between the positions where the tight binding threads 6 are wound and the like.

For example, silk, cotton, polyethylene terephthalate resin (PET), nylon, rubber, vinyl resin, and PEEK resin can be used as materials for forming the tight binding threads 6.

The cured adhesive body 7 is formed by curing the thermosetting adhesive for a medical device in which the infrared absorbent is diffused into the thermosetting resin. The cured adhesive body 7 is configured to cover and come in close contact with the outer circumferential surface 5a of the coating tube 5 and the tight binding threads 6 at least at the part where the tight binding threads 6 are wound such that the outer circumferential surface 5a and the tight binding threads 6 are bonded with each other.

The coating tube 5 and the tight binding threads 6 are the bonded members which are bonded by the cured adhesive body 7.

According to the present embodiment, the cured adhesive body 7 is formed in a longitudinal direction of the insertion portion 2 such that the distal end side comes in contact with the proximal end part of the distal-end cover 4 and the proximal end side is formed in a circular region covering the proximal end side of the tight binding threads 6. Together with the outer side surface of the distal-end cover 4 and the outer circumferential surface 5a of the coating tube 5, the cured adhesive body 7 is configured to form part of the outer circumferential surface at the distal end part of the insertion portion 2.

A method of forming the cured adhesive body 7 will be described.

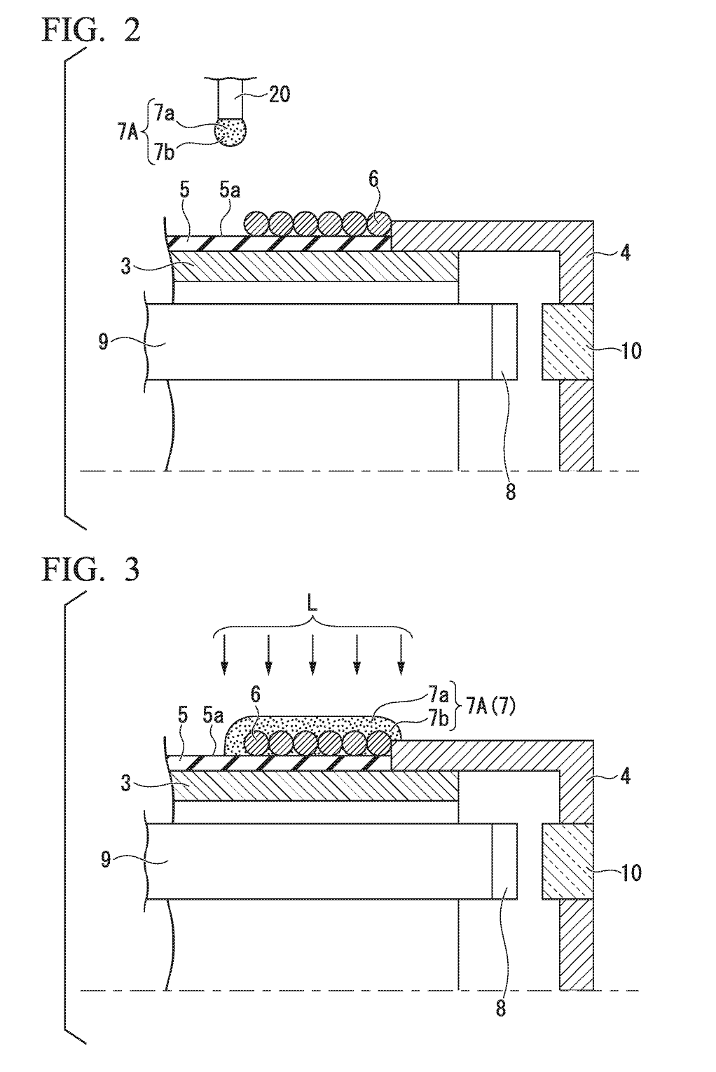

FIG. 2 is a schematic partial cross-sectional view showing a coating procedure of a thermosetting adhesive for a medical device during manufacture of the medical device according to the embodiment of the present invention. FIG. 3 is a schematic partial cross-sectional view showing a curing procedure of the thermosetting adhesive for a medical device during manufacture of the medical device according to the embodiment of the present invention.

In order to form the cured adhesive body 7, as shown in FIG. 2, after winding the tight binding threads 6 on the outer circumferential surface 5a of the coating tube 5 externally fitted with the adaptor 3, a thermosetting adhesive (thermosetting adhesive for a medical device) 7A is coated on the coating tube 5 and the tight binding threads 6 (coating procedure).

The thermosetting adhesive 7A is the thermosetting adhesive for a medical device which is cured by the irradiation of infrared light.

The thermosetting adhesive 7A has an adhesive main body 7a and an infrared absorbent 7b.

The adhesive main body 7a has a thermosetting resin and a curing agent configured to cure the thermosetting resin by being heated.

The thermosetting resin included in the adhesive main body 7a only has to be the thermosetting resin that can be used in the adhesive portion of the medical device and it not particularly limited.

Suitable resin materials can be adopted as the thermosetting resin included in the adhesive main body 7a in accordance with the materials of the bonded members as bonding targets, the position at which the cured adhesive body 7 is disposed, and the like.

For example, as shown in FIG. 1, when the cured adhesive body 7 is disposed on the outer circumferential surface of the distal end part of the endoscope apparatus 1, resin materials having biocompatibility so as not to cause any problem even if the cured adhesive body 7 comes in contact with living tissues, and heat resistance and chemical resistance so as to withstand sterilization procedures can be used.

For example, resin materials that can be used as the thermosetting resin of the adhesive main body 7a may be one of epoxy resin, silicone resin, urethane resin, and acrylic resin, or a resin material formed with one of these derivatives described above as a main component.

Epoxy resin, silicone resin, acrylic resin, and phenol novolac resin are more preferable as the thermosetting resin of the adhesive main body 7a for the endoscope apparatus 1.

Suitable curing agent included in the adhesive main body 7a is selected in accordance with the type of the thermosetting resin. For example, the curing agent included in the adhesive main body 7a can be amine, dimethylamine, amide, dimethylamide, amide derivative, ether amine, and the like.

As necessary, the adhesive main body 7a may have other additive besides the thermosetting resin and the curing agent. For example, the adhesive main body 7a may have inorganic particles such as silica, alumina, and the like, and the adhesive main body 7a may have organic materials such as primer, curing accelerator, and the like.

A material having a maximum absorption peak in the infrared region among a wavelength range of the visible region (400 nm-700 nm) and the infrared region (700 nm-1 mm) is adopted as the infrared absorbent 7b. Hereinafter, a wavelength of the maximum absorption peak in the infrared region is referred to a maximum absorption wavelength.

The infrared absorbent 7b can be liquid or solid. When the infrared absorbent 7b is solid, the infrared absorbent 7b may be suitably formed in a powdered shape, a particle shape, a fibrous shape, and the like.

The infrared absorbent 7b may be formed by mixing various different types of materials. When the infrared absorbent 7b is formed by various materials with different types, any of a type of the compounds, a type of the maximum absorption wavelengths, a type of phase states such as solid, liquid and the like, a type of shapes such as a powered shape, a particle shape, a fibrous shape, and the like, and a type of dimensions such as a particle diameter and the like may be different.

The infrared absorbent 7b is diffused into the thermosetting resin of the thermosetting adhesive 7A as an additive.

The infrared absorbent 7b is configured to transform light energy of the infrared light into thermal energy by absorbing the infrared light passing through the thermosetting resin.

A specific material of the infrared absorbent 7b may be infrared light absorbent dyes.

The infrared absorbent 7b may include at least one of a group consisting of cyanine compound, phthalocyanine compound, dithiol metal complex, naphthoquinone compound, diimonium compound, and azo compound.

A content amount of the infrared absorbent 7b with respect to the adhesive main body 7a only has to be determined so as to efficiently perform photothermal conversion and is not particularly limited. For example, when the content amount of the thermosetting resin and the curing agent is determined to be 10 parts by weight, the infrared absorbent 7b may be included by equal to or more than 1 part by weight, and equal to or less than 3 parts by weight.

However, when the infrared absorbent 7b is included by less than 1 part by weight, since the too little infrared light is absorbed by the infrared absorbent 7b, it will take a lot of time for curing.

When the infrared absorbent 7b is included by more than 3 parts by weight, it will lead to deterioration in the operability of coating the adhesive.

A method of coating the thermosetting adhesive 7A is not particularly limited. For example, as shown in FIG. 2, the thermosetting adhesive 7A may be coated on the coating tube 5a and the tight binding threads 6 by being ejected by a dispenser 20.

As shown in FIG. 3, when the cured adhesive body 7 is coated to cover the tight binding threads 6, the coating procedure is finished.

Next, the thermosetting adhesive 7A is cured by heating the thermosetting adhesive 7A (curing procedure).

In the present embodiment, the thermosetting adhesive 7A is heated by an irradiation of the infrared light L toward the area where the thermosetting adhesive 7A is coated.

It is more preferable that the wavelength of the infrared light L includes the maximum absorption wavelength of the infrared absorbent 7b.

Alight source irradiating the infrared light L may be an infrared laser source or an infrared LED, also may be an infrared lamp. Furthermore, an infrared heater may be used to since the infrared heater also irradiates the infrared light L.

It is preferable that the infrared light L irradiates a limited area in which the thermosetting adhesive 7A is coated.

When the infrared light L irradiates the thermosetting adhesive 7A, the infrared light L is absorbed by the thermosetting adhesive 7A. The infrared absorbent 7b is included in the thermosetting adhesive 7A such that the infrared light L is efficiently absorbed by the infrared absorbent 7b. Accordingly, the thermosetting adhesive 7A is efficiently heated.

The infrared absorbent 7b diffused into the thermosetting adhesive 7A absorbs the infrared light L to heat the thermosetting adhesive 7A also from the inside of the thermosetting adhesive 7A such that a curing time of the thermosetting adhesive 7A is shortened.

Furthermore, compared to a situation when the infrared absorbent 7b is not included, an amount of the infrared light L that is transmitted or reflected by the thermosetting adhesive 7A is reduced such that an amount of the infrared light L that is irradiated toward other members besides the thermosetting adhesive 7A is reduced.

As a result, temperature increase in other members besides the thermosetting adhesive 7A can be suppressed.

For example, electronic components and electronic circuits such as the image sensor 8 and the driving circuit thereof are disposed at the distal end part of the insertion portion 2. If such electronic components and electronic circuits used in the endoscope apparatus 1 is applied with an intense heat, it will lead to a damage or a deterioration due to the thermal stress.

According to the thermosetting adhesive 7A of the present embodiment, an amount of heat applied to the members disposed at the outside of the coating area of the thermosetting adhesive 7A is reduced such that the damage and deterioration of the electronic components and the electronic circuits disposed in the vicinity of the coating area due to the thermal stress can be prevented.

When the thermosetting adhesive 7A is cured, the cured adhesive body 7 is formed to come in close contact with at least the coating tube 5 and the tight binding threads 6. Accordingly, the coating tube 5 and the tight binding threads 6 are bonded with each other via the cured adhesive body 7. A surface of the tight binding threads 6 are covered by the cured adhesive body 7 such that the tight binding threads 6 are protected. Accordingly, a state in which the coating tube 5 is fixed to the adaptor 3 by the tight binding threads 6 is stably maintained.

Furthermore, the cured adhesive body 7 is configured to form part of the outer circumferential surface of the distal end part of the insertion portion 2 such that the uneven surface due to the tight binding threads 6 is smoothed and the sliding characteristic of the insertion portion 2 is improved.

As described above, since the infrared absorbent 7b is diffused into the thermosetting adhesive 7A according to the present embodiment, the thermosetting adhesive 7A is heated by the irradiation of the infrared light and rapidly cured.

In the endoscope apparatus 1 according to the present embodiment, the bonded members are bonded by the cured adhesive body 7 which is cured by irradiating the thermosetting adhesive 7A with the infrared light. During the curing procedure of the thermosetting adhesive 7A, the thermosetting adhesive 7A is rapidly and definitely cured even if the infrared light L locally irradiates the coating area of the thermosetting adhesive 7A. Accordingly, the temperature increase of the members disposed at the outside of the coating area of the thermosetting adhesive 7A is suppressed.

According to the endoscope apparatus 1, it is possible to reduce the affection of the thermal stress when the thermosetting adhesive 7A is heated and shorten the manufacturing time.

As a result, durability and life span of the endoscope apparatus 1 is improved. Further, manufacturing cost of the endoscope apparatus 1 is reduced.

In the description of the present embodiment, an example of using the thermosetting adhesive 7A to bond the coating tube 5 and the tight binding threads 6 wound on the distal end part of the insertion portion 2 is described.

However, the bonding area and the bonded members due to the thermosetting adhesive 7A are not limited thereto.

For example, the thermosetting adhesive 7A may also be used at the proximal end part of the insertion portion 2 to bond the coating tube 5 and tight binding threads for fixing the coating tube 5 to the adaptor.

In the description of the present embodiment, an example of coating the thermosetting adhesive 7A on the most outside circumference of the medical device and curing the thermosetting adhesive 7A is described. If the irradiation of the infrared light with respect to the thermosetting adhesive 7A is applicable during the manufacturing, the thermosetting adhesive 7A may be coated on any portion of the medical device including an interior portion thereof.

In the description of the present embodiment, an example of the endoscope apparatus 1 as the medical device is described. However, variations of the medical device according to the present embodiment of the present invention is not limited to the endo scope apparatus. For example, embodiments of the medical device of the present invention may be medical devices such as a treatment tool, a catheter, a balloon, and the like.

In the description of the present embodiment, an example of forming part of the most outside portion of the insertion portion 2 using the cured adhesive body 7 is described. A layered portion for improving the sliding characteristic and hydrophilicity may be formed on the outer circumferential surface of the cured adhesive body 7.

In the description of the present embodiment, the infrared absorbent is defined as the material having the maximum absorption peak in the infrared region among the wide wavelength range including the visible region (400 nm-700 nm) and the infrared region (700 nm-1000 nm). However, since there is a possibility that the infrared light cannot transmit through the main agent depending on the wavelength, it is preferable that the infrared absorbent is a material having the maximum absorption peak in the near-infrared region among the wavelength range including the visible region (400 nm-700 nm) and the near-infrared region (700 nm-110 nm). Taking the wavelength of the infrared illumination light source into consideration, it is more preferable that the infrared absorbent has the maximum absorption peak in a wavelength of 750 nm-1000 nm.

### EXAMPLES

Next, examples of the present embodiment will be described together with comparison examples.

In the following Table 1, compositions of the thermosetting adhesives for medical device used in the examples 1-9 and comparison examples 1, 2, and evaluation results thereof will be shown.

TABLE 1

| | COMPOSITION | | | |
|---|---|---|---|---|
| | MAIN AGENT + CURING AGENT | | | ADDITIVE AGENT |
| | MAIN AGENT MATERIAL | CURING AGENT MATERIAL | WEIGHT PART | MATERIAL |
| EXAMPLE 1 | EPOXY RESIN | ALIPHATIC AMINE | 10 | INFRARED ABSORBENT #1 |
| EXAMPLE 2 | EPOXY RESIN | ALIPHATIC AMINE | 10 | INFRARED ABSORBENT #1 |
| EXAMPLE 3 | EPOXY RESIN | ALIPHATIC AMINE | 10 | INFRARED ABSORBENT #1 |
| EXAMPLE 4 | EPOXY RESIN | ALIPHATIC AMINE | 10 | INFRARED ABSORBENT #1 |
| EXAMPLE 5 | EPOXY RESIN | ALIPHATIC AMINE | 10 | INFRARED ABSORBENT #2 |
| EXAMPLE 6 | EPOXY RESIN | ALIPHATIC AMINE | 10 | INFRARED ABSORBENT #3 |
| EXAMPLE 7 | EPOXY RESIN | ALIPHATIC AMINE | 10 | INFRARED ABSORBENT #4 |
| EXAMPLE 8 | EPOXY RESIN | ALIPHATIC AMINE | 10 | INFRARED ABSORBENT #5 |
| EXAMPLE 9 | EPOXY RESIN | ALIPHATIC AMINE | 10 | INFRARED ABSORBENT #6 |
| EXAMPLE 10 | EPOXY RESIN | ALIPHATIC AMINE | 10 | INFRARED ABSORBENT #7 |
| EXAMPLE 11 | EPOXY RESIN | ALIPHATIC AMINE | 10 | INFRARED ABSORBENT #8 |
| EXAMPLE 12 | EPOXY RESIN | ALIPHATIC AMINE | 10 | INFRARED ABSORBENT #9 |
| COMPARISON | EPOXY RESIN | ALIPHATIC AMINE | 10 | CARBON BLACK |
| EXAMPLE 1 | | | | |
| COMPARISON | EPOXY RESIN | ALIPHATIC AMINE | 10 | CARBON BLACK |
| EXAMPLE 2 | | | | |

| | COMPOSITION | | | |
|---|---|---|---|---|
| | ADDITIVE AGENT | EVALUATION | | |
| | WEIGHT PART | CURING METHOD | CURING TIME (MINUTES) | OPERATIVITY |
| EXAMPLE 1 | 1 | INFRARED RADIATION | 8 | ◯ |
| EXAMPLE 2 | 2 | INFRARED RADIATION | 5 | ◯ |
| EXAMPLE 3 | 3 | INFRARED RADIATION | 5 | ◯ |
| EXAMPLE 4 | 5 | INFRARED RADIATION | 5 | Δ |
| EXAMPLE 5 | 2 | INFRARED RADIATION | 5 | ◯ |
| EXAMPLE 6 | 2 | INFRARED RADIATION | 5 | ◯ |
| EXAMPLE 7 | 2 | INFRARED RADIATION | 5 | ◯ |
| EXAMPLE 8 | 2 | INFRARED RADIATION | 5 | ◯ |
| EXAMPLE 9 | 2 | INFRARED RADIATION | 5 | ◯ |
| EXAMPLE 10 | 2 | INFRARED RADIATION | 5 | ◯ |
| EXAMPLE 11 | 2 | INFRARED RADIATION | 5 | ◯ |
| EXAMPLE 12 | 2 | INFRARED RADIATION | 5 | ◯ |
| COMPARISON | 2 | DRYING FURNACE | 80 | ◯ |
| EXAMPLE 1 | | | | |
| COMPARISON | 2 | INFRARED RADIATION | 30 | ◯ |
| EXAMPLE 2 | | | | |

Maximum absorption wavelengths (shown as “maximum wavelength” in Table 2) of additives in Table 1, and brand names and manufacturers of additives in Table 1 are shown in Table 2.

TABLE 2

| | MAXIMUM | | |
|---|---|---|---|
| | WAVELENGTH | | |
| | (nm) | BRAND NAME | MANUFACTURER |
| INFRARED ABSORBENT #1 | 780 | KP Deeper NR Paste | NIPPON KAYAKU CO., LTD. |
| INFRARED ABSORBENT #2 | 817 | IR-T | SHOWA DENKO K. K. |
| INFRARED ABSORBENT #3 | 819 | IR-13F | SHOWA DENKO K. K. |
| INFRARED ABSORBENT #4 | 780 | YKR-2016 | YAMAMOTO CHEMICALS, INC. |
| INFRARED ABSORBENT #5 | 790 | YKR-2100 | YAMAMOTO CHEMICALS, INC. |
| INFRARED ABSORBENT #6 | 900 | YKR-2900 | YAMAMOTO CHEMICALS, INC. |
| INFRARED ABSORBENT #7 | 890 | YKR-2081 | YAMAMOTO CHEMICALS, INC. |
| INFRARED ABSORBENT #8 | 1000 | YKR-2200 | YAMAMOTO CHEMICALS, INC. |
| INFRARED ABSORBENT #9 | 840 | YKR-2090 | YAMAMOTO CHEMICALS, INC. |
| CARBON BLACK | — | AT-NO. 40 | ORIENTAL INDUSTRY CO.LTD. |

### Example 1

As shown in Table 1, in the thermosetting adhesive 7A of Example 1, the adhesive main body 7a is formed by using bisphenol F-type epoxy resin as a main component and aliphatic amine as a curing agent.

Specifically, ADEKA RESIN (registered trade-mark) EP-4901 (brand name; manufactured by ADEKA Corporation) is used as the bisphenol F-type epoxy resin. Epoxy resin curing agent ST-13 (brand name; manufactured by Mitsubishi Chemical Corporation) is used as the curing agent.

The thermosetting adhesive 7A of the Example 1 is configured to include the main component and the curing agent by a sum of 10 parts by weight and infrared absorbent #1 is added by 1 part by weight. It is known that a mixing ratio of the man component and the curing agent does not significantly affect the curing time. In the present example, for example, the main component is included by 6 parts by weight and the curing agent is included by 4 parts by weight.

As shown in Table 2, a dispersion liquid type of infrared absorbent KP Deeper NR Paste (brand name; manufactured by Nippon Kayaku Co., Ltd.) is used at the infrared absorbent #1. The maximum absorption wavelength with respect to the infrared light of the infrared absorbent #1 is 780 nm.

A method of irradiating the thermosetting adhesive 7A with the infrared light having a wavelength of 800 nm is adopted as a curing method for the thermosetting adhesive 7A of Example 1. Irradiation amount of the infrared light is set to be 500 W.

### Examples 2-4

As shown in Table 1, the thermosetting adhesives 7A of the Examples 2-4 are different from the thermosetting adhesive 7A of the Example 1 in that the infrared absorbent #1 of the Example 1 is added by 2, 3, and 5 parts by weight, respectively.

### Examples 5-12

As shown in Table 1, the thermosetting adhesives 7A of the Examples 5-12 are different from the thermosetting adhesive 7A of the Example 2 in that infrared absorbent #2-#9 are added to the thermosetting adhesives 7A of the Examples 5-12 respectively, instead of the infrared absorbent #1 of the Example 2.

Furthermore, since the maximum absorption wavelengths of the infrared absorbents are different from each other, wavelengths of the infrared light irradiated for curing the thermosetting adhesive according to Examples 5-12 are appropriately changed.

Next, difference from the Example 2 will be mainly described.

As shown in Table 2, NIR-photosensitive dyes IR-T (brand name; manufactured by Showa Denko K.K.) is used as the infrared absorbent #2 of Example 5. The maximum absorption wavelength of the infrared absorbent #2 is 817 nm. The wavelength of the infrared light used for curing is 800 nm.

NIR-photosensitive dyes IR-13F (brand name; manufactured by Showa Denko K.K.) is used as the infrared absorbent #3 of Example 6. The maximum absorption wavelength of the infrared absorbent #3 is 819 nm. The wavelength of the infrared light used for curing is 800 nm.

NIR-photosensitive dyes YKR-2016 (brand name; manufactured by Yamamoto Chemicals, Inc.) is used as the infrared absorbent #4 of Example 7. The maximum absorption wavelength of the infrared absorbent #4 is 780 nm. The wavelength of the infrared light used for curing is 800 nm.

NIR-photosensitive dyes YKR-2100 (brand name; manufactured by Yamamoto Chemicals, Inc.) is used as the infrared absorbent #5 of Example 8. The maximum absorption wavelength of the infrared absorbent #5 is 790 nm. The wavelength of the infrared light used for curing is 800 nm.

NIR-photosensitive dyes YKR-2900 (brand name; manufactured by Yamamoto Chemicals, Inc.) is used as the infrared absorbent #6 of Example 9. The maximum absorption wavelength of the infrared absorbent #6 is 900 nm. The wavelength of the infrared light used for curing is 800 nm.

NIR-photosensitive dyes YKR-2081 (brand name; manufactured by Yamamoto Chemicals, Inc.) is used as the infrared absorbent #7 of Example 10. The maximum absorption wavelength of the infrared absorbent #7 is 890 nm. The wavelength of the infrared light used for curing is 800 nm.

NIR-photosensitive dyes YKR-2200 (brand name; manufactured by Yamamoto Chemicals, Inc.) is used as the infrared absorbent #8 of Example 11. The maximum absorption wavelength of the infrared absorbent #8 is 1000 nm. The wavelength of the infrared light used for curing is 1000 nm.

NIR-photosensitive dyes YKR-2090 (brand name; manufactured by Yamamoto Chemicals, Inc.) is used as the infrared absorbent #9 of Example 12. The maximum absorption wavelength of the infrared absorbent #9 is 840 nm. The wavelength of the infrared light used for curing is 800 nm.

### Comparison Example 1

As shown in Table 1, the thermosetting adhesive of the Comparison Example 1 is different from that of the Example 2 in that instead of the infrared absorbent #1 of Example 2, carbon black is added by the same parts by weight as the infrared absorbent #1 of Example 2.

Specifically, as shown in FIG. 2, amorphous-type graphite powder AT-No. 40 (brand name; manufactured by Oriental Industry Co. Ltd.) is used as the carbon black material.

Further, in the Comparison Example 1, a method of heating by a drying oven is performed as a curing method. The heating temperature by the drying oven is set at 80 degrees Celsius.

### Comparison Example 2

As shown in Table 1, Comparison Example 2 is different from the Comparison Example 1 only in that a method of irradiating with the infrared light is adopted as the curing method.

Since the carbon black of the Comparison Example 1 does not have a maximum absorption wavelength with respect to the infrared light, the wavelength and the irradiation amount of the infrared light are set to be the same as that of the Example 2 described above.

### Evaluation

As an evaluation, measurements of curing time of the thermosetting adhesive and evaluations of operability of coating operations according to each Example and each Comparison Example are performed.

The operability of the coating operations is evaluated by judging easiness of the coating operations by the operator by three grades, for example, a good grade (shown as “∘” in Table 1), a fair grade (shown as “Δ” in Table 1), and a poor grade (shown as “x” in table 1). The “fair grade” refers to a situation in which the coating operation can be performed, however, compared with the “good grade”, it is necessary to perform the coating operation carefully such that the operation time increases. The “poor grade” refers to a situation in which viscosity of the thermosetting adhesive is significantly high such that a comfortable result is not achieved even if a lot of time is spent to perform the coating operation carefully.

### Evaluation Results

As shown in the column “curing time” in Table 1, with regard to the curing time of the Examples 1-4 which were different from each other only in the addition amount of the infrared absorbent #1, the curing time of the Example 1 is 8 minutes and the curing time of the Examples 2-4 were 5 minutes.

Accordingly, if the addition amount of the infrared absorbent #1 was equal to or more than 2 parts by weight, there was no difference in the curing time. Even if the addition amount of the infrared absorbent #1 was 1 part by weight as the Example 1, the curing time was only increased by 3 minutes.

With regard to the operability, the Examples 1-3 were evaluated by the “good grade”, and the Example 4 was evaluated by the “fair grade”. In the Example 4, the addition amount of the infrared absorbent #1 was 5 parts by weight such that the viscosity of the thermosetting adhesive 7A increases and it became difficult to perform the coating operation. However, if the coating operation was performed carefully, coating unevenness was suppressed and an acceptable level was achieved.

A relationship between the addition amount of the infrared absorbent, the curing time, and the operability was not significantly changed even when the type of the infrared absorbent was changed. Accordingly, the addition amounts of the infrared absorbent in the Examples 5-12 were set to be the same parts by weight with that in the Example 2.

The curing time in each of the Examples 5-12 was 5 minutes, and the operability in each of the Examples 5-12 was the “good grade”.

Compared with the above, in the Comparison Example 1 in which the thermosetting adhesive was cured by the drying oven, the heating temperature was set at 80 degrees Celsius in order to prevent bad affection to the electronic components, the curing time was extended to 80 minutes.

In the Comparison Example 2 in which the carbo black was added instead of the infrared absorbent, the curing time was 30 minutes. This was because the carbon black was inferior to the infrared absorbents #1-#9 in the absorption characteristic of the infrared light.

According to the evaluation results described above, compared with the Comparison Examples 1 and 2, it can be seen in the Examples 1-12 that the curing time was significantly shortened.

The embodiments of the invention have been described above with reference to the drawings, but specific structures of the invention are not limited to the embodiments and may include various modifications without departing from the scope of the invention. The invention is not limited to the above-mentioned embodiments and is limited only by the accompanying claims.

What is claimed is:

1. A medical device comprising:
a bonded member, and
a cured adhesive body in close contact with the bonded member,
wherein the cured adhesive body is formed by curing a thermosetting adhesive for a medical device, and
wherein the thermosetting adhesive for a medical device is formed by diffusing an infrared absorbent into a thermosetting resin.

2. The medical device according to claim 1,
wherein the bonded member comprises
a flexible coating member configured to coat a base member for fixing; and
tight binding threads being wound on an outer surface of the coating member, and
wherein the cured adhesive body is configured to cover and come in close contact with the tight binding threads and the outer surface of the coating member.

3. The medical device according to claim 1, wherein the infrared absorbent includes at least one of a group consisting of a cyanine compound, a phthalocyanine compound, a dithiol metal complex, a naphthoquinone compound, a diimonium compound, and an azo compound.

4. A thermosetting adhesive for a medical device being cured by an irradiation of infrared light, comprising:
a thermosetting resin; and
an infrared absorbent diffused into the thermosetting resin.

5. The thermosetting adhesive for a medical device according to claim 4, wherein the infrared absorbent includes at least one of a group consisting of a cyanine compound, a phthalocyanine compound, a dithiol metal complex, a naphthoquinone compound, a diimonium compound, and an azo compound.
